(19) United States
(12) Patent Application Publication
(10) Pub. No.: US 2004/0225188 A1
(43) Pub. Date: Nov. 11, 2004

(54) Apparatus and method for implementing an endoscopic marker

(76) Inventors: Martin Kleen, Neunkirchen (DE); Rainer Kuth, Herzogenaurach (DE)

Correspondence Address: HARNESS, DICKEY & PIERCE, P.L.C., P.O.BOX 8910, RESTON, VA, 20195, US

(21) Appl. No.: 10/824,384

(22) Filed: Apr. 15, 2004

(30) Foreign Application Priority Data
Apr. 15, 2003 (DE) 10317368.4

(51) Int. Cl. A61B 1/015

(52) U.S. Cl. 600/159; 600/118; 600/109

(57) ABSTRACT

A wireless endoscope unit includes an RF transmitter for transmitting and receiving signals via an antenna, and an arrangement for taking individual images of the surroundings. These images are then transferred via the RF transmitter to an external image processing unit. In this case, a control serves for executing control commands received via the RF receiver. A dye container is included for keeping a dye and an exit opening, connected to the dye container, is for implementing the dye in tissue in accordance with the control commands of the control.

The present application hereby claims priority under 35 U.S.C. §119 on German patent application number DE 103 17 368.4 filed Apr. 15, 2003, the entire contents of which are hereby incorporated herein by reference.

### FIELD OF THE INVENTION

The invention generally relates to a wireless endoscope unit, and/or to a method for operating a wireless endoscope unit.

### BACKGROUND OF THE INVENTION

An endoscope that takes individual images of the surroundings and transmits them to an external image processing system is used to examine the upper or lower gastrointestinal tract. Lesions, for example tumors, can be identified and localized with the aid of the images. However, during subsequent examinations or interventions it is often difficult to find the lesion again, since there are no landmarks available in the intestine, which is up to 11 m long.

In addition to the use of endoscopy in the region of the gastrointestinal tract, numerous further possible applications are available or are being planned. What is involved in general is the examination of cavities in the interior of the body. This can be, for example, the examination of the abdomen via a small incision at the navel, the examination of the lungs by means of a pencil-slim endoscope, or of the blood vessels.

Moreover, given a high level of sterility, for example during stereotactic operations, thin instruments can be introduced into the brain. This can be done via small bore holes for biopsies or minimally invasive therapies.

A device for a wireless endoscope unit in the form of a swallowable capsule has already been disclosed by DE 2929429, published after the priority date of the present application. Here, there is integrated in the endoscope a permanent magnet which can control the position of the endoscope by use of a magnetic field applied from outside. The endoscope unit uses an objective lens and a CCD chip to take individual images of the surroundings and transfer them together with position data to an external image processing system.

However, this device has the disadvantage that no markings can be implemented in lesions to facilitate finding the latter again during subsequent examinations.

A further known method is chromoendoscopy. Here, in the case of examinations of the esophagus, for example, the mucous membrane is stained with harmless dyes by a spray catheter such that changes are clearly contrasted and can thus be better detected.

However, it is a disadvantage here that the dyes serve merely for better identification of lesions during the endoscopic examination.

Also known are invasive techniques for marking, in which, for example, guide wires are installed at a lesion under the control of imaging diagnostic systems.

Document WO 00/22975 discloses a swallowable capsule for taking images of the gastrointestinal tract, and for setting markings or for taking tissue samples. For this purpose, the capsule has a stretchable container, in which a dye or a marking device is contained, and a stretchable diaphragm, on one side of the container, which can be pressed into or withdrawn from the stretchable container by a reversibly extendable spring which can be contracted again. The dye located in the container is thereby pressurized.

A tip reaching into the dye container is located on the inner side of the diaphragm. By extending the spring the tip punctures the opposite side of the stretchable container so that the dye contained in the container can emerge. It is possible in this way to apply a dose of dye once.

### SUMMARY OF THE INVENTION

An object of an embodiment of the present invention is to reduce or even eliminate at least one of the disadvantages of the prior art.

An object may be achieved according to an embodiment of the invention by a device and/or a method.

In accordance with an embodiment of the present invention, a wireless endoscope unit includes an RF transmitter/RF receiver for transmitting and receiving signals via an antenna, an arrangement for taking individual images of the surroundings, which are transferred via the RF transmitter to an external image processing unit, a control for executing the control commands received via the RF receiver, a dye container for keeping a dye, and an exit opening, connected to the dye container, for implementing the dye in tissue in accordance with the control commands of the control. In this case, the dye container may be connected to the exit opening via an operating channel, and a repeatedly actuable closure may be integrated in the operating channel.

Moreover, according to an embodiment of the present invention, a method for a wireless endoscope unit includes: receiving and transmitting signals via an RF transmitter/RF receiver and an antenna, taking individual images of the surroundings and transferring these images to an external image processing unit via the RF transmitter, executing control commands received via the RF transmitter, keeping a dye in a dye container, and implementing the dye in tissue via an exit opening, connected to the dye container, in accordance with the received control commands. Here, the method may include connecting the dye container to the exit opening via an operating channel, and/or integrating a repeatedly actuable closure in the operating channel.

Owing to the use of a control which obtains control commands received via an antenna, it is possible to execute commands for implementing the dye during the endoscopic examination. The use of a dye which is kept in a dye container integrated in the endoscope unit permits a marker to be applied noninvasively or minimally invasively at a lesion for the purpose of identification during subsequent interventions or examinations, for example during a surgical operation. It is often very difficult, after opening the abdominal wall during the actual operation, to find the previously endoscopically localized lesion again. This is because there are no landmarks available on the intestine, which is up to 11 m long.

### BRIEF DESCRIPTION OF THE DRAWINGS

The present invention will become more fully understood from the detailed description of exemplary embodiments given hereinbelow and the accompanying drawings, which are given by way of illustration only and thus are not limitative of the present invention, and wherein:

FIG. 1 shows a schematic of a wireless endoscope unit in accordance with an embodiment of the invention,

FIG. 2 shows a first embodiment of the device for implementing an endoscopic marker,

FIG. 3 shows a second embodiment of the device for implementing an endoscopic marker, and

FIG. 4 shows a third embodiment of the device for implementing an endoscopic marker.

### DETAILED DESCRIPTION OF THE PREFERRED EMBODIMENTS

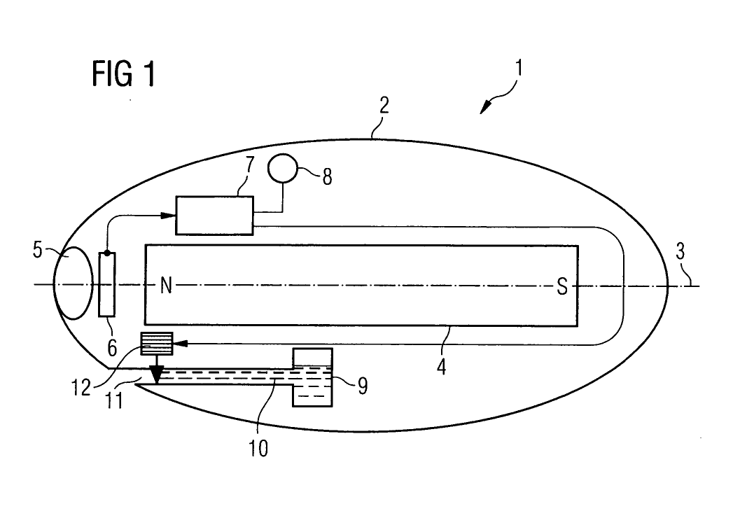

The first step below is to describe the mode of operation of an embodiment of the inventive wireless endoscope unit with reference to FIG. 1. What is involved here is a wireless endoscope unit 1 in the form of a swallowable capsule. The housing 2 is made from a biocompatible material that is resistant to digestive secretions occurring in the gastrointestinal tract. A permanent magnet 4 that is installed in the housing along a fixed longitudinal axis 3 permits the endoscope unit to be aligned from outside by an external applied magnetic field. The capsule progresses through the gastrointestinal tract owing to the peristaltic movements of the gastrointestinal musculature.

Individual images of the surroundings, for example the inner wall or other tissue of the gastrointestinal tract of a body, are taken by an imaging device, such as an objective lens 5 with a downstream CCD chip 6 for example. At a rate of two images per second, the capsule supplies approximately 57 000 images during the eight-hour process of examination.

An RF transmitter/RF receiver 7 with an antenna 8 may serve as a transceiver to transmit the images taken by the objective lens 5 and the CCD chip 6 to, for example, an external image processing unit. Moreover, via the antenna 8 for example, the RF transmitter/RF receiver 7 may receive control commands from an external command unit, and may transfer these to a control 12 integrated in the endoscope unit 1.

Again, integrated in the endoscope unit 1 is a dye container 9 for keeping a dye that is intended to be used to implement a marking in tissue. The dye container 9 is connected in this case via an operating channel 10 to an exit opening 11 via which the dye emerges. Integrated in the operating channel 10 is a closure 13 which prevents the uncontrolled emergence of the dye.

It is possible, furthermore, to integrate several dye containers 9 into the endoscope unit that can be controlled individually from the outside, and which in each case permit a dose of dye to be applied once or repeatedly.

The dye used is a nonpoisonous dye, for example a dye whose staining action can be achieved with a dose whose toxicity is appropriately related to the clinical benefit. The dye can be a solution, an emulsion, a suspension or a mixture thereof, and includes particles which penetrate into the inner surface or mucous membrane of the acantha and have a half residence time there of at least several hours.

It is possible in this case to use substances other than dye, depending on the further investigations or interventions that are planned. It is possible, for example, to use a fluorescing dye that fluoresces in the visible region when illuminated in the ultraviolet. Outstanding visibility even of very small quantities is thereby possible, for example in the case of surgical interventions for removing the lesion previously identified and marked by the endoscope unit.

A further possibility is the use of a dye that achieves a high contrast in the imaging method, these including, for example, GdDTPA or iron particles for X-ray pictures or MRI (magneto resonance imaging). Moreover, the dye can be a substance that can be detected with the aid of a simple sensor in a very small concentration, for example a substance with a high magnetic susceptibility μ.

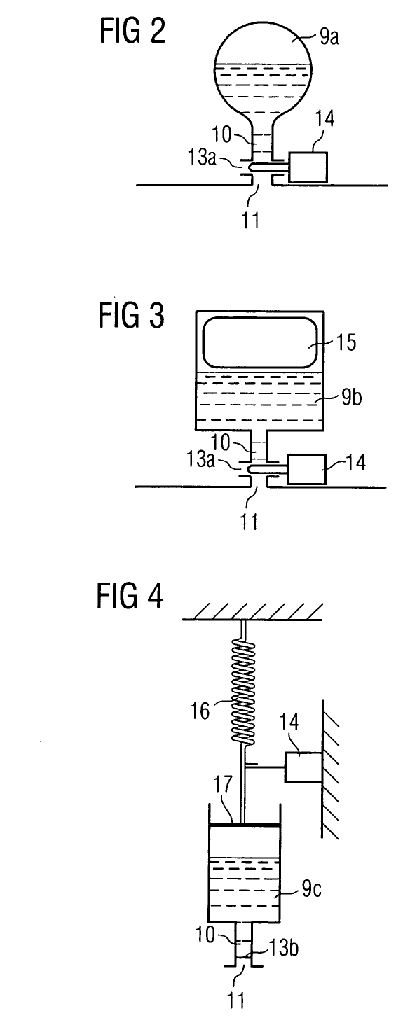

A first embodiment of the inventive device is to be explained with the aid of FIG. 2. The dye container 9a is in this case a stretchable rubber diaphragm that is mechanically pressurized after being filled with dye. The closure 13 is here a valve 13a that is opened and closed by a piezoelectric crystal 14.

By applying a voltage or a voltage pulse, the piezoelectric crystal 14 contracts, the valve 13a is opened, and the pressure of the filled stretchable rubber diaphragm 9a causes the dye to emerge via the exit opening 11. If there is no longer any voltage across the piezoelectric crystal 14, the latter expands again and the valve 13 is closed, thus stopping the application of dye. If the dye has not emerged completely from the dye container 9a, it is possible for a further dose of dye to be applied by applying a renewed voltage pulse to the piezoelectric crystal 14.

In a second exemplary embodiment (compare FIG. 3), the dye container 9 includes a rigid container 9b in which an elastic air container 15 is located. The air container 15 is surrounded by an elastic skin and is under overpressure. The result is that pressure is exerted in the rigid container 9b on the dye reservoir located therein.

As in FIG. 2, the closure 13 is a valve 13a that is opened and closed as appropriate via a piezoelectric crystal 14. If the piezoelectric crystal contracts, the valve 13a opens and the dye emerges via the exit opening 11 owing to the pressure produced by the elastic air container 15 in the rigid container 9b. If there is no longer any voltage across the piezoelectric crystal 14, the latter re-expands and the valve 13a is closed, the result being to stop the application of dye. If a voltage is reapplied across the piezoelectric crystal 14, the valve 13a reopens, and further dye can emerge from the container 9b for the purpose of applying a dose of dye once more.

The dye container 9 in a third embodiment of the invention in accordance with FIG. 4 is a rigid container 9c that has a displaceable plunger 17 on one side. The movement of the displaceable plunger 17 is regulated via a spring 16. If the spring 16 is in the compressed state, the plunger 17 is at the edge of the rigid container 9c, and if the spring 16 expands a pressure is exerted on the displaceable plunger 17, the result being that the latter is moved into the rigid container 9c, in which the dye is located, and thus presses the dye out.

In the initial state, the spring 16 is in the compressed state, and is held by a piezoelectric crystal 14. After application of a voltage or a voltage pulse to the piezoelectric crystal 14, the latter contracts and releases the spring 16, as a result of which the spring 16 expands and exerts pressure on the displaceable plunger 17, and thus on the dye contained in the rigid container 9c.

Here, the closure 13 integrated in the operating channel 10 is a diaphragm 13b which automatically ruptures under pressure. If pressure is thus exerted on the dye reservoir owing to the displacing of the plunger 17, the diaphragm 13b ruptures and the dye emerges. In this embodiment, it is possible for each dye container 9c to apply a dose of dye only once, and not repeatedly.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A wireless endoscope device, comprising:
an RF transmitter/RF receiver for transmitting and receiving signals via an antenna;
an arrangement for taking individual images of surroundings, which are transferable via the RF transmitter to an external image processing unit;
a control for executing control commands received via the RF receiver/RF transmitter;
a dye container for holding a dye; and
an exit opening, connected to the dye container, through which the dye is implemented in tissue in accordance with the control commands of the control, wherein the dye container is connected to the exit opening via an operating channel, and wherein a repeatedly actuable closure is integrated in the operating channel.

2. The device as claimed in claim 1, wherein the dye container is a stretchable rubber diaphragm.

3. The device as claimed in claim 1, wherein the dye container is a rigid container.

4. The device as claimed in claim 3, wherein an elastic air container is integrated in the rigid container.

5. The device as claimed in claim 1, wherein the closure is a valve.

6. The device as claimed in claim 1, wherein a piezoelectric crystal controls the opening and closing of the closure.

7. A method for operating a wireless endoscope unit, comprising:
receiving and transmitting signals via an RF transmitter/RF receiver and an antenna;
taking images of surroundings and transferring the images to an external image processing unit via the RF transmitter;
executing control commands received via the RF transmitter;
maintaining a dye in a dye container; and
implementing the dye in tissue via an exit opening, connected to the dye container, in accordance with the received control commands, wherein the dye container is connected to the exit opening via an operating channel, and wherein a repeatedly actuable closure is integrated into the operating channel.

8. The device as claimed in claim 5, wherein a piezoelectric crystal controls the opening and closing of the closure.

9. The device as claimed in claim 2, wherein the closure is a valve.

10. The device as claimed in claim 2, wherein a piezoelectric crystal controls the opening and closing of the closure.

11. The device as claimed in claim 4, wherein the closure is a valve.

12. The device as claimed in claim 11, wherein a piezoelectric crystal controls the opening and closing of the closure.

13. The device as claimed in claim 4, wherein a piezoelectric crystal controls the opening and closing of the closure.

14. The method as claimed in claim 7, wherein a piezoelectric crystal controls the opening and closing of the closure.

15. The method as claimed in claim 7, wherein the dye container is a stretchable rubber diaphragm.

16. The method as claimed in claim 7, wherein the dye container is a rigid container.

17. The method as claimed in claim 16, wherein an elastic air container is integrated in the rigid container.

18. A wireless endoscope device, comprising:
an imaging device for imaging body tissue; and
a dye container for holding a dye, wherein a device is connected to the dye container for permitting the dye to escape to mark the body tissue.

19. The device as claimed in claim 18, wherein the dye container includes a stretchable rubber diaphragm.

20. The device as claimed in claim 18, wherein the dye container includes a rigid container.

21. The device as claimed in claim 19, wherein an elastic air container is integrated in the rigid container.

22. The device as claimed in claim 18, wherein the device connected to the dye container includes a valve.

23. The device as claimed in claim 18, wherein a piezoelectric crystal controls opening of the device connected to the dye container, to permit the dye to escape and mark the body tissue.

24. The device as claimed in claim 22, wherein a piezoelectric crystal controls opening and closing of the valve.

25. The device as claimed in claim 22, wherein the valve is voltage controlled.

26. The device as claimed in claim 18, wherein opening of the device connected to the dye container is controlled using voltage, to permit the dye to escape and mark the body tissue.

27. The device as claimed in claim 18, wherein voltage controls opening and closing of the device connected to the dye container.

28. The device as claimed in claim 23, wherein the dye container includes a stretchable rubber diaphragm.

29. The device as claimed in claim 23, wherein the dye container includes a rigid container.

30. The device as claimed in claim 29, wherein an elastic air container is integrated in the rigid container.

31. The device as claimed in claim 24, wherein the dye container includes a stretchable rubber diaphragm.

32. The device as claimed in claim 24, wherein the dye container includes a rigid container.

33. The device as claimed in claim 32, wherein an elastic air container is integrated in the rigid container.

34. A wireless endoscope device, comprising:
means for imaging body tissue;
means for holding a dye; and
means for permitting the dye to escape to mark the body tissue.

35. The device as claimed in claim 34, wherein the means for holding the dye includes a stretchable rubber diaphragm.

36. The device as claimed in claim 34, wherein the means for holding the dye includes a rigid container.

37. The device as claimed in claim 36, wherein an elastic air container is integrated in the rigid container.

38. The device as claimed in claim 34, further comprising:
means for controlling opening of the means for holding the dye, to permit the dye to escape and mark the body tissue.

39. The device as claimed in claim 38, wherein voltage controls opening and closing of the means for holding the dye.

40. The device as claimed in claim 34, further comprising:
means for controlling opening and closing of the means for holding the dye based upon voltage.

41. The device as claimed in claim 38, wherein a piezoelectric crystal controls opening and closing of the means for holding the dye.

42. A method of operating a wireless endoscope device, comprising:
imaging body tissue;
holding a dye in a container of the wireless endoscope device; and
permitting the dye to escape to thereby mark the body tissue.

43. The method as claimed in claim 42, the step of permitting includes controlling an opening and closing of a device connected to the container.

44. The method as claimed in claim 43, wherein the controlling is achieved via a piezoelectric device.

45. The method as claimed in claim 42, wherein the dye is held in a stretchable rubber diaphragm.

46. The method as claimed in claim 42, wherein the dye is held in a rigid container.

47. The method as claimed in claim 46, wherein an elastic air container is integrated in the rigid container.
